(19) United States
(12) Patent Application Publication
(10) Pub. No.: US 2025/0259736 A1
(43) Pub. Date: Aug. 14, 2025

(54) SYSTEMS AND METHODS FOR ENHANCED EXTENDED CARE MANAGEMENT WITH SOCIAL DETERMINANTS OF HEALTH

(71) Applicant: ClinNEXUS Inc., Los Angeles, CA (US)

(72) Inventors: Gregory Shawn CARROLL, Los Angeles, CA (US); Peter Andrew CROSBY, Aliso Viejo, CA (US); Kevin Michael FICKENSCHER, Kittery, ME (US)

(73) Assignee: ClinNEXUS Inc., Los Angeles, CA (US)

(21) Appl. No.: 18/892,119

(22) Filed: Sep. 20, 2024

Related U.S. Application Data
(60) Provisional application No. 63/552,160, filed on Feb. 11, 2024.

(51) Int. Cl. G16H 40/00; G16H 10/60

(52) U.S. Cl. CPC G16H 40/00; G16H 10/60

(57) ABSTRACT

A method and system for providing enhanced extended care management of patients which may include a service selection module that selects services to be provided from a menu of services, based on patient characteristics and Prioritization of services based on patient characteristics and prior data from patient outcomes.

### I. CROSS-REFERENCE TO RELATED APPLICATION

This application is related to, and claims the benefit of, U.S. Provisional Patent Application No. 63/552,160, filed Feb. 11, 2024, the entire contents of which are incorporated herein by reference.

### II. FIELD OF THE INVENTION

The present disclosure relates generally to the field of patient management and continuity of care delivery by health care organizations. It also relates to integration of Social Determinants of Health (SDOH) into extended care management.

### III. BACKGROUND OF THE INVENTION

Hospital readmission is a huge problem. Patients discharged from hospital with many different conditions and who do not receive timely, effective, and responsive care following discharge have an increased incidence of relapse frequently requiring readmission which leads to adverse outcomes and elevated financial costs. Historically, nearly 20% of all Medicare patients discharged from hospital had a readmission within 30 days (Beauvais, Bradley, et al. “Is the Hospital Value-Based Purchasing Program Associated with Reduced Hospital Readmissions?” Journal of Multidisciplinary Healthcare (2022): 1089-1099). This results in billions of dollars in potentially avoidable costs.

These readmission rates and resulting additional costs were confirmed by the Agency for Health Care Research and Quality with application to all the major payors including Medicare, Medicaid, and private insurers (Weiss A J (IBM Watson Health), Jiang H J (AHRQ). Overview of Clinical Conditions With Frequent and Costly Hospital Readmissions by Payer, 2018. HCUP Statistical Brief #278. July 2021. Agency for Healthcare Research and Quality, Rockville, MD1).

Hospital readmission is a particularly troublesome financial problem within a health care payment system of a fixed fee per enrolled patient per month (PEPM)—so called value-based reimbursement, Examples include Medicare Advantage and, in most US states, Medicaid. It is in the financial interest of health care providers to minimize hospital readmissions.

The US government (via the Centers for Medicare and Medicaid Services or CMS) established the Hospital Readmissions Reduction Program (HRRP). Section 1886(q) of the Social Security Act sets forth the statutory requirements for HRRP, which requires the Secretary of the U.S. Department of Health and Human Services to reduce payments under subsection (d) hospitals for excess readmissions beginning Oct. 1, 2012. CMS includes the following condition or procedure-specific 30-day risk-standardized unplanned readmission measures in the program:
Acute myocardial infarction (“AMI”)
Chronic obstructive pulmonary disease (“COPD”)
Heart failure (“HF”)
Pneumonia
Coronary artery bypass graft (“CABG”) surgery
Elective primary total hip arthroplasty and/or total knee arthroplasty (“THA” and “TKA”)

By 2021, over 90% of acute-care hospitals had been penalized at least once in the past decade for excess readmissions through the HRRP. This is a clear indication that present efforts to reduce hospital readmissions are inadequate. Furthermore, the academic literature clearly denotes that the hospital readmission problem extends beyond the six major post-discharge problems included in the HRRP requirements to include many other clinical problems.

By 2016, it was reported (Zuckerman, Rachael B., et al. “Readmissions, observation, and the hospital readmissions reduction program.” New England Journal of Medicine 374.16 (2016): 1543-1551) that readmission rates for the targeted conditions declined from 21.5% to 17.8%. Although statistically significant and helpful, this is not enough to make meaningfully large savings to the nation's healthcare costs, and more needs to be done.

It is not just hospital readmission that is a huge problem, the problem starts with prevention of admission of patients with chronic conditions in the first place. Many patients with chronic conditions (e.g.: heart failure, kidney failure, diabetes) do not receive adequate care and are at risk of the condition deteriorating to the point that they need to be admitted to a hospital for treatment. Hospital admissions can be reduced, the overall health of the population can be improved, and costs can be reduced by providing comprehensive health care at home or in society at large to minimize hospital admissions.

Maternal mortality is defined as death while pregnant or within 42 days of the end of pregnancy. The maternal mortality rate in the US at 17.4 per 100,000 live births is about twice that of comparable developed countries like France (8.7) and Canada (8.6) and 10 times higher than New Zealand (1.7) (Maternal Mortality and Maternity Care in the United States Compared to 10 Other Developed Countries (Commonwealth Fund, November 2020), https://doi.org/10.26099/411v-9255). At risk women (especially those living in poverty or marginalized) often lack access to maternity care providers, a support network, housing, adequate nutrition, and comprehensive post-partum support. Maternal mortality may be reduced by providing pre-natal and post-partum care and a network of midwives to assist with birth.

In many cases, hospital admission or readmission, and maternal mortality outcomes are driven by Social Determinants of Health (“SDOH”) which historically have not been well integrated into the US health care system. SDOH includes factors not generally counted in the category of “health care” such as nutrition, poverty, housing, transport, social and family support, medication delivery, and access to advice.

Several publications point out the value of integrated information that addresses SDOH, but a comprehensive system that incorporates clinical information and SDOH has not been described. There is no comprehensive and accepted method of incorporating SDOH into patient care. For example, Garg et al (Garg, A., Homer, C. J., & Dworkin, P. H. (2019). Addressing social determinants of health: Challenges and opportunities in a value-based model. In Pediatrics (Vol. 143, Issue 4). American Academy of Pediatrics) pointed out the need for addressing SDOH in a pediatric population but provided no guidance on how it should be implemented. Similarly, the importance of SDOH has been established in diabetes management (e.g.: Hill-Briggs, F., Adler, N. E., Berkowitz, S. A., Chin, M. H., Gary-Webb, T. L., Navas-Acien, A., Thornton, P. L., & Haire-Joshu, D. (2021). Social determinants of health and diabetes: A scientific review. In Diabetes Care (Vol. 44, Issue 1, pp. 258-279) and heart failure (White-Williams, C., Rossi, L. P., Bittner, V. A., Driscoll, A., Durant, R. W., Granger, B. B., Graven, L. J., Kitko, L., Newlin, K., & Shirey, M. (2020). Addressing Social Determinants of Health in the Care of Patients with Heart Failure: A Scientific Statement from the American Heart Association. Circulation, 141 (22), e841-e863).

The US government has recognized the importance of SDOH and states “that clinical care impacts only 20 percent of county-level variation in health outcomes, while social determinants of health (SDOH) affect as much as 50 percent. Within SDOH, socioeconomic factors such as poverty, employment, and education have the largest impact on health outcomes” (Whitman, A., de Lew, N., Chappel, A., Aysola, V., Zuckerman, R., & Sommers, B. D. (n.d.). Addressing Social Determinants of Health: Examples of Successful Evidence-Based Strategies and Current Federal Efforts” (Whitman, Amelia, et al. “Addressing social determinants of health; Examples of successful evidence-based strategies and current federal efforts.” Office Health Policy 1 (2022): 1-30 www.aspe.hhs.gov/sites/default/files/documents/e2b650cd64cf84aae8ff0fae7474af82/SDOH-Evidence-Review.pdf).

Transition Care Management (“TCM”) refers to intervention with at-risk patients during in-hospital treatment or at or soon after hospital discharge, often with the goal of reducing hospital readmission. TCM has demonstrated improved patient outcomes in many dimensions, including hospital readmissions. A related program, Extended Care Management (“ECM”) is a collaborative and interdisciplinary approach to the provision of services to certain patients with complex needs that hamper the ability of the patient and the patient's care providers (e.g.: physicians, nurses, other health care professionals, family, in-home help) to improve their health. While TCM is focused on a particular short-term outcome (hospital readmission), ECM is targeted at longer-term outcome measures. Both ECM and TCM are programs that may be financially supported by health schemes such as Medicare and Medicaid (administered by the states).

Finlayson (Finlayson, Kathleen, et al. “Transitional care interventions reduce unplanned hospital readmissions in high-risk older adults.” BMC health services research 18 (2018): 1-9.) conducted a prospective randomized clinical trial that showed that multifaceted transitional care interventions across hospital and community settings help to reduce hospital readmission rates in the first 12 weeks post-discharge in an Australian setting. Those patients receiving exercise, nurse home visit, and telephone follow-up were reported as having a 13% unplanned readmission rate at 28 days, compared to 25% in the control group. While these data are encouraging, the study did not provide insights on the best method of customizing transitional care intervention based on the patient characteristics, or any other methodology, or describe a system for implementing TCM.

Herberg and Teuteberg (Herberg, S., & Teuteberg, F. (2023). Reducing hospital admissions and transfers to long-term inpatient care: A systematic literature review. In Health Services Management Research (Vol. 36, Issue 1, pp. 10-24)) conducted a systematic literature review that noted that there is great number of variations in care management programs, and there is a need for a standardized approach, particularly for an elderly population in a rural environment.

Similarly, Baldino (Baldino, Michael, et al. “Impact of a novel post-discharge transitions of care clinic on hospital readmissions.” Journal of the National Medical Association 113.2 (2021): 133-141) reported that a group of 373 patients given an in-person appointment at the Transitions of Care Clinic within 14 days of discharge showed a statistically significant reduction in 30-day readmission rates from 15.3% for the control group to 8.85% for the intervention group. While this study emphasizes the importance of transition care management, it did not show the value of a transition care management program with integrated virtual and in-person intervention and ongoing support nor describe a method of implementing TCM. In particular, the authors noted that many participants were lost to follow-up, and therefore the results are skewed because the intervention group only included those who adhered to the protocol.

The Baldino publication, perhaps unwittingly, pointed out one of the limitations of traditional ECM and TCM systems—and that is the lack of follow-up of patients in the community. Similarly, Baldino, like other publications, does not give any insight into the challenges of enrolling patients into an ECM or TCM system.

Rasmussen (Rasmussen, Lisa Fønss, et al. “Impact of transitional care interventions on hospital readmissions in older medical patients: a systematic review.” BMJ open 11.1 (2021): e040057) conducted a systematic review and found that “the majority of interventions in the transitional phase between hospital and home appears to reduce readmission rates among older patients discharged from a medical ward.” Again, the authors did not describe a system or method for providing TCM.

Kash (Kash, Bita A., et al. “Successful hospital readmission reduction initiatives: Top five strategies to consider implementing today.” Journal of Hospital Administration 7.6 (2018): 16-23) conducted a comparative study that showed that the most successful strategies were:
Collaboration between clinical teams and/or community providers
Post-discharge home visits
Telephone follow-up calls
Patient and/or family education
Discharge planning

The authors noted that “The top five strategies with the highest impact on reducing readmission rates are all collaborative in nature and would likely benefit from a values-based medical practice model. Operationalizing values-based medical practice requires a multi-faceted and systematic approach to healthcare delivery redesign.” The authors did not describe a system or method for selecting which intervention(s) would be most appropriate for which patients, nor how such interventions should be delivered, nor the impact of such interventions.

The use of Artificial Neural Networks (“ANN”), Artificial Intelligence (“AI”), Machine Learning (“ML”) and or Deep Learning (“DL”) holds promise for augmenting a system to provide ECM or TCM. In this description, ANN, AI, ML and DL are referred to collectively as AI.

Huang (Huang, Yinan, et al. (“Application of machine learning in predicting hospital readmissions: a scoping review of the literature.” BMC medical research methodology 21.1 (2021): 1-14) performed a literature review on the use of electronic health records or population records with ML to predict hospital readmissions. The majority of those studies reported the Area Under the Curve (“AUC”) of the Receiver Operating Characteristics (“ROC”) curve above 0.70, representing modest to high discrimination ability. However, Huang provided no insight as to how the ML systems could go beyond merely predicting hospital readmissions to using the prediction models to customize an intervention for reducing the risk of hospital readmission.

Liu (Liu, Wenshuo, et al. “Predicting 30-day hospital readmissions using artificial neural networks with medical code embedding.” PloS one 15.4 (2020): e0221606) showed that AI methods can improve on simple regression-based modeling to predict hospital readmissions for four diseases, but the improvements were modest (e.g.: AUC from 0.60 to 0.64 for heart failure).

Dixit (Dixit, Rohit R. “Risk Assessment for Hospital Readmissions: Insights from Machine Learning Algorithms.” Sage Science Review of Applied Machine Learning 4.2 (2021): 1-15.) performed a retrospective analysis of data from 130 US hospitals for the period 1999-2008 to explore different AI approaches for predicting hospital readmissions. While this analysis highlighted the potential usefulness of AI, the authors acknowledged the limitations of the study particularly the exclusions of several factors such as severity of illness and SDOH which are crucial factors that could affect readmission rates, and the authors did not describe a system or method for implementing an AI system to be used in practice.

These approaches to predicting the risk of readmission do not account for how interventions, if any, change the risk of readmission. Nor does merely predicting the risk of readmission guide action. There remains an unmet need for a system and method to determine the risk of readmission after intervention, and adjusting interventions based on the recalculated risk.

There remains an unmet need for providing a system and method to provide ECM and TCM with the goal of minimizing unplanned hospital readmission based on a model used to predict the risk of hospital readmission in which interventions are customized to a particular patient's circumstances.

There is an unmet need for a system and method for providing ECM and TCM that accounts for a wider spectrum of factors for predicting risk of subsequent events (including hospital readmission), which uses individual, community and population outcome data to refine the model and guide interventions selected.

There is an unmet need for a system and method providing ECM and TCM that recommends a sequence of interventions for best patient outcomes based on prior data and then tracks the outcomes from those interventions.

There is an unmet need for a system and method for guiding delivery of ECM and TCM to pregnant women to reduce the risk of maternal and infant mortality.

There is an unmet need for a system and method for guiding ECM and TCM interventions which does not require complete data and will perform almost as well with a sparse dataset as a complete dataset.

There is an unmet need for a system and method for providing ECM and TCM that actively reaches out for patient enrolment and active follow-up.

The present invention is a comprehensive system and method targeted at all at-risk patients to reduce complications, including hospital admissions and readmissions, through the provision of Transition Care Management (TCM) and enhanced Extended Care Management (ECM), with an emphasis on SDOH. Provision of TCM and ECM has the potential of reducing health care costs, improving health and quality of life of the population, and extending healthy lives.

A manual proof-of-concept system to explore the value of TCM was used within a Medicaid environment in California (the Medi-Cal Health Care Program). The manual proof-of-concept system was for the purpose of showing the value of TCM and did not use automation elements as described in the present invention. While the manual system showed the value of TCM, it is not scalable to a larger population.

Over a four-month period, there were 399 patients who qualified for TCM services. Of those, 328 (82.2%) were enrolled. Of those, 249 patients completed at least one face to face visit and 131 patients (52.6%) completed the TCM program. Of those 131 patients, there were 7 readmissions within 30 days of enrolment, for a readmission rate of 5.3%.

These results compare well with the historical readmission rate in a comparable patient population in the same geographical region of 14.8%.

### IV. PRIOR ART

There are several publications on systems and methods for predicting the risk of hospital readmission, based on patient attributes.

For example, U.S. Pat. No. 10,943,676 to Farooq et a (the '676 patent) discloses a computer-based system for predicting the probability of a given patient to be readmitted. The probability can be predicted during a patient's hospital stay and used to generate workflow action items to reduce the probability of readmission. In this system, the patient's hospital records are used as the primary data source for data mining for predicting readmission to a particular medical entity such as a hospital. The '676 provides no teaching or insight into how to include other, non-traditional healthcare data to improve outcomes.

The '676 invention does not disclose an apparatus or method for adjusting or recalculating the risk of readmission based on interventions and therefore has limited use in clinical practice.

There are some publications that describe systems and methods for automating patient care.

For example, U.S. Pat. No. 10,665,334 (Baniameri et al, May 26, 2020—the '334 patent) describes a Method and System for Automated Healthcare Care Coordination and Care Transitions. The method includes selecting a patient for care coordination based on patient diagnosis and data analytics, and then assigning a predetermined patient care plan, based on several inputs and then customizing the care plan in response to input from a healthcare provider or the patient. However, the '334 patent does not teach how to incorporate crucial elements from the in-home and social elements of the patient's environment which would limit effectiveness.

Automated patient care may include delivery of care at a distance—“telehealth.” For example, U.S. Pat. No. 10,325,070 (Beale and Jaruzel, Jun. 18, 2019—the '070 patent) describes a computer system and methods for providing precision psychotherapy or other mental health interventions customized to the patient and served at a distance.

The '070 patent discloses an AI system that is continually learning such that one set of patient experiences is used to train the AI system to refine future recommendations for the therapist delivering the therapy via telehealth. The '070 patent is limited to providing remote interventions via a healthcare professional, and does not include provision of any other services that might be advantageous to the patient's outcome.

U.S. Pat. No. 11,551,792 to Stadler et al (the '792 patent) describes a cloud-based computer system to receive and process health data from disparate sources for identifying, stratifying and prioritizing patients eligible for care management services. This invention is targeted to identifying patients, and does not describe a system or method for actually providing care management services.

U.S. Pat. No. 10,957,451 to Satyanarayan and Gogulamudi (the '451 patent) describes an apparatus and method for communication between a patient and a health care system to recommend a treatment plan, in which the recommendations my be generated by an AI system, and the recommendations may be updated based on the outcomes of the initial treatment plan. The '451 patent does not describe integration with SDOH. nor does it describe how the patient interacts with a care management system.

### V. BRIEF DESCRIPTION OF THE DRAWINGS

FIG. 1 illustrates the elements that interact to provide ECM or TCM.

FIG. 2 illustrates a block diagram of a system and method to implement the present invention.

FIG. 3 is a representation of one embodiment of organization of the Central Service Provider.

FIG. 4 illustrates a representation of data flows between the Health Care Provider and the Central Service Provider.

FIG. 5 illustrates an example of organization of an ANN.

FIG. 6 illustrates an example of prioritized selection from a menu of services.

FIG. 7 illustrates the flowchart of activities for a typical patient.

### VI. SUMMARY OF THE INVENTION

The present invention provides a system and methods for providing Transition Care Management and enhanced Extended Care Management, with an emphasis on SDOH.

### VII. DETAILED DESCRIPTION OF THE INVENTION

The detailed description of the exemplary embodiments and examples herein are by way of illustration only and are not limiting on the scope of the invention. A person of skill in the art will understand that changes can be made without departing from the spirit and scope of this disclosure.

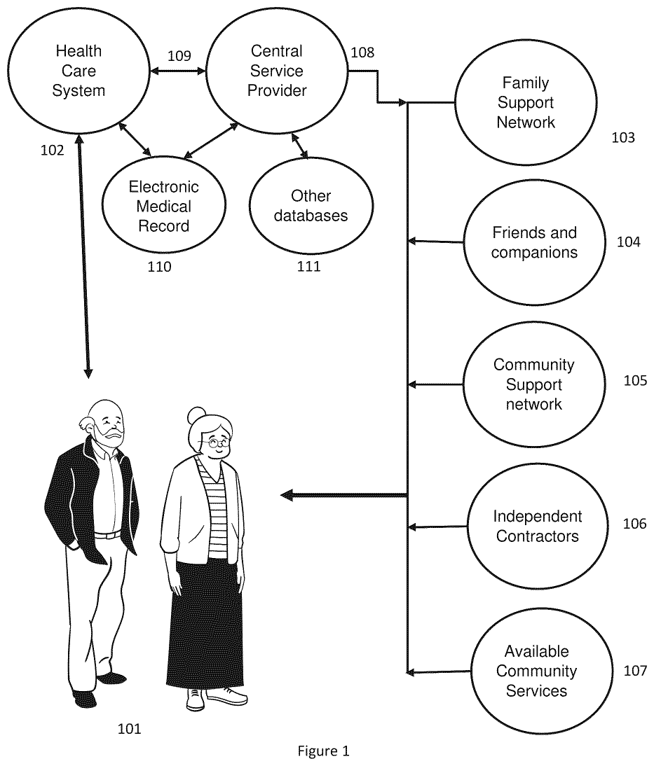

By way of example, the elements in the network of services that interact to provide ECM and/or TCM are illustrated in FIG. 1. There may be other elements not mentioned in this description and omission of those other elements is not intended to be limiting on the invention.

Patients 101 are the focus of the system. Patients interact with the Health Care System (“HCS”) 102, which may include hospitals, outpatient clinics, family care physicians, specialist physicians, pharmacies, urgent care facilities, aged care facilities, nursing homes, in-home visits from a health care professional (“HCP”), and the like. The HCS 102 generally uses an Electronic Medical Record (“EMR”) 110. The system is configured to access other databases 111 for information not generally available in the EMR and may also receive data entered manually.

Other elements of the community which are not normally considered as part of the health care system can impact the patient's outcomes and quality of care. These elements contribute to the SDOH which have been shown to have a major impact on a patient's health and well-being. See for example, Braverman, Paula, Susan Egerter, and David R. Williams. “The social determinants of health: coming of age.” Annual review of public health 32 (2011): 381-398. It has been suggested that provision of mobile health aligned with the SDOH would provide better outcomes (Rogers, C. C., Jang, S. S., Tidwell, W., Shaughnessy, S., Milburn, J., Hauck, F. R., Williams, I. C., & Valdez, R. S. (2023). Designing mobile health to align with the social determinants of health. Frontiers in Digital Health, 5, https://doi.org/10.3389/fdgth.2023.1193920).

These elements may include the family support network 103 (co-resident, local, or extended family), friends and companions 104 (which may include animal companions and pets), the community support network 105 (which may include churches, volunteer and charitable services such as meals on wheels), independent contractors 106 which provide paid services as requested (e.g.: transportation services (taxis, Uber), food delivery services, house cleaning and laundry services, physical therapists, broad band internet and mobile phone service), and available community services 107 such as social workers, translation services, employment assistance, and tax help.

Although most communities have at least some of the elements, few communities have all the elements. Many patients have limited knowledge of available services, often there is poor coordination of services and unclear mechanisms to provide (and pay) for services to those who need them. In this invention, a Central Service Provider (“CSP”) 108 is configured to coordinate delivery of services to provide ECM and TCM. The CSP may provide some services directly (e.g.: telephone follow-up of a patient) and may initiate and coordinate the provision of services from the community elements or the HCS

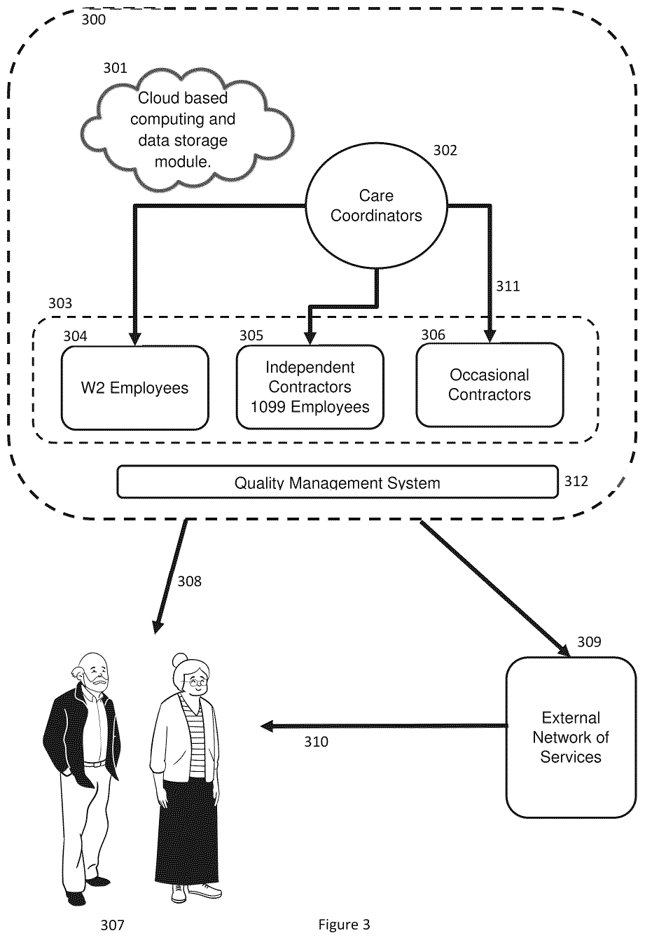

The organization of the CSP is illustrated in FIG. 3, described below in detail. In summary, the CSP consists of as organization of people who coordinate the provision of services.

The CSP interacts and communicates 109 with the HCS. For example, the HCS may request or contract with the CSP to provide ECM or TCM services to the patient(s). The CSP may provide information back to the HCS about patient outcomes, arrange scheduling, and provide information about the patient's health care outside the HCS e.g.: in another hospital while traveling. The CSP may interact with the HCS EMR 110 to take data about the patients, or to update the EMR (and/or other databases 111) with patient outcomes and history of services provided.

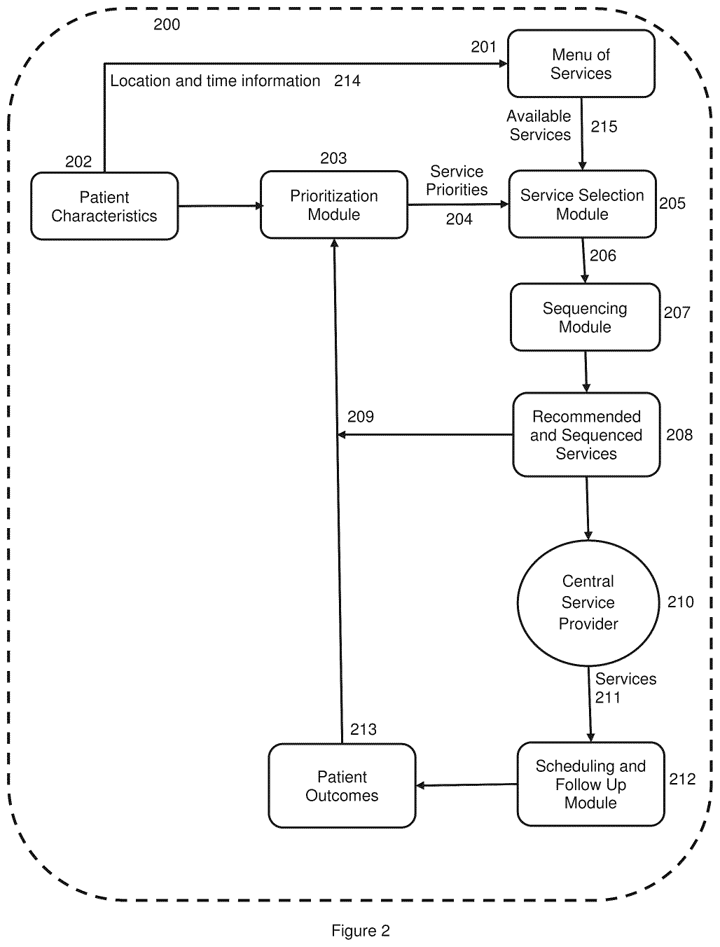

In this invention, the CSP 200 is organized to provide ECM and TCM as illustrated in the block diagram in FIG. 2.

A menu of services 201 is provided. In a preferred embodiment, the menu of services 201 includes at least some of the services described above and illustrated in FIG. 1, which generally fit into the categories of clinical services and social services. Clinical services might include in-home visits by an HCP; telephone consultation with an HCP; counseling; scheduling hospital or clinic visits; drug delivery to the home; and other services. Social services might include arranging transportation (e.g.: for a clinic visit); language and translation services; in-home food delivery; family education; referral to a social worker; and others. As time goes on, additional services may be added to the menu of services, or obsolete services can be removed from the menu. In this manner, the menu of services is continually updated to reflect the best possibilities.

Each element in the menu of services is tagged to reflect geographical and time availability. Thus the available services 215 may be different for each patient and for different times of day. For example, some patients may be in an area in which social workers are not available, so social workers would excluded in the services available for that patient.

Patient characteristics include clinical characteristics such as diagnosis, duration of current clinical condition, prior treatments, medications, comorbidities (e.g.: diabetes, obesity, hypertension), history, physical characteristics (height, weight, age, BMI), and vital signs. Many of these clinical characteristics may be obtained from the hospital patient records either automatically (computer-to-computer interface for the EMR), manual entry, or some combination. Patient characteristics might also include social characteristics such as marital status, ethnicity, employment status, family support, languages spoken, access to transportation, analysis of family or community support systems, access to nutrition, and income level. Patient characteristics may also include annotations from providers such as clinicians, nurses, and medical assistants.

The EMR is not the sole source of information on the needs of patients. The invention described herein incorporates the ability to retrieve data from other databases 111 with which it interfaces. Examples of such databases include employment, social services,, home nursing care program, family members, or other external entity engaged in elements of care delivery for the patient.

Some of the patient's characteristics may need to be entered manually.

Patient characteristics can include categorical variables (e.g.: sex) and continuous variables (e.g.: height). Patient characteristics can also include derivative variables e.g.: whether or not the patient's blood glucose is within or outside the normal range for that patient.

Key elements of patient characteristics are location information and time. Location can be both a permanent location (i.e.: place of residence) or a current location (e.g.: when working away from the home). Current location information can be provided, for example by a mobile phone which interacts with the system. As described above, Location and Time Information 214 modifies the menu of services to reflect local availability so the Available Services 215 for that patient at that time are one input to the Service Selection Module 205. Location information is also used in other ways as will be described below.

Patient Characteristics 202 is one set of inputs to the Prioritization Module 203. The Prioritization Module generates numerical Service Priorities to the menu of services to prioritize which services should be provided. The Prioritization module uses a computer system (which may be local or a cloud based computer system, or a distributed system) and uses one or more algorithms or, in a preferred embodiment, an AI engine to generate the Priorities.

Prioritization Module 203 generates numerical Service Priorities 204 to the Service Selection Module 205 which uses the priorities and the Menu of Services 201 to select one or more services to be provided to the patient. The Service Priorities are used to prioritize services based on analysis of the patient's individual data from Patient Characteristics 202 and other inputs e.g.: historical data, location and time. The historical data could include data aggregated from multiple patients that have used the system, as well as more general population statistics, and any other source of data that could be used to train an AI engine. Historical data may include data from each individual patient. As will be appreciated, the more factors that are used in the Prioritization module to determine Priorities to prioritize services, the more robust the system will be to missing data.

The Service Selection Module uses the Service Priorities 204 and Available Services 215 to provide Recommended Services 206 which are communicated to a Sequencing Module 207. The sequencing module uses the recommended services and integrates with other information such as the availability of selected services (e.g.: a clinic visit must fit in with the clinic schedule) and information about if and how sequencing of services makes a difference to the outcome, among other factors. For example, prior information about a particular patient may show that the particular patient usually needs two reminder phone calls to attend a clinic visit, as well as arranging transportation, whereas another particular patient needs only one reminder via text message and has her own transportation, or another patient requires communication and integration of family members in assisting the patient with support. The Service Selection Module 205 and the Sequencing Module 207 thus work together to optimize the services to be provided for the best outcomes at minimal cost, tailored to each individual patient.

The recommended and sequenced services for a particular patient 208 are communicated 209 back to the Prioritization module 203 which maintains a record of services for each patient. The recommended and sequenced services are also communicated to the CSP 210 which coordinates the provision of services as illustrated in FIG. 1 and described in detail eow with reference to FIG. 3. As the services 211 are provided, a Scheduling and Follow-up Module 212 keeps track of services provided (or attempted to be provided), and patient outcomes.

From time to time, the patient is followed up coordinated by a Scheduling and Follow-up Module 213. In a preferred embodiment, at least some of the follow-up could be automatic, for example a text message with a reminder for the patient to take medications or schedule a clinic visit. In a further embodiment, follow-up could be in person e.g.: with a clinic visit at the discharging hospital or another healthcare provider, a telephone follow-up, or an in-home follow-up. In a further embodiment, follow-up could be automatic e.g.: with a home blood pressure monitor (systolic and diastolic blood pressure, and heart rate) or bathroom scales (patient weight) that connects (e.g.: via Wi-Fi or the cell phone system) to automatically provide follow-up data.

The outcomes from follow-up are input to a Patient Outcomes module 208 which accumulates and aggregates the follow-up data. The patient outcomes are an input to the Prioritization module 203 which uses those outcome data (among others) to adjust the Prioritization for the service selection module 205. Patient Outcomes may be communicated 109 back to the HCS 102 in real time or occasionally (batch mode).

Information about the recommended and sequenced services 208 is one set of inputs to the Prioritization Module 203. In some cases, the services recommended might not be provided e.g.: because a patient misses a follow-up visit, so it is important that the Prioritization Module 203 has inputs of the services actually provided, not just those recommended. For example, if a patient routinely misses clinic follow-up visits, the Prioritization module could take that into account to de-prioritize clinic follow-up visits and emphasize telephone follow-up. The information about services actually provided is provided by the Patient Outcomes Module 213.

In a preferred embodiment, the Prioritization module 203 incorporates AI such that the Service Selection Module is informed by the prior experience of a large set of patients, as well as the individual outcomes of each patient. The AI engine in the Prioritization module 203 can be any of the structures well-known to those skilled in the art. An AI system can incorporate multiple inputs and outputs with accuracy and time beyond the capabilities of any human or group of humans.

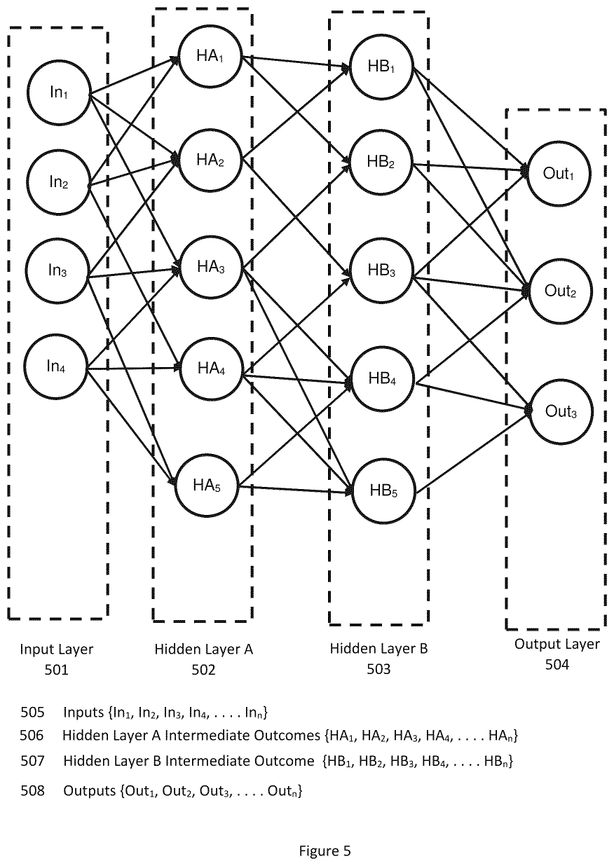

An example of one embodiment of an AI based Prioritization module is shown in FIG. 5. This is a two-layer artificial neural network (ANN) with an input layer 501, two hidden layers 502 and 503, and an output layer 504. As will be appreciated by one skilled in the art, an ANN may have any number of hidden layers, and number of inputs and outputs. The inputs to the input layer, {In₁, In₂, In₃, In₄, . . . Inₙ} include Patient Characteristics 202, Patient Outcomes 213, and recommended and sequenced services 209. The outputs of the ANN {Out₁, Out₂, Out₃, . . . Outₙ} are the Priorities which are the input to the Service Selection Module 205. The connections between the layers are for illustration only, and a person skilled in the art will understand that there can be more or fewer connections between the nodes in each layer.

The intermediate outcomes in the hidden layer A 502 {HA₁, HA₂, HA₃, HA₄, . . . HAₙ} depend on the inputs and the weights between the inputs and the intermediate outcomes that arise from the training of the ANN. Similarly, the intermediate outcomes in the hidden layer B 503 {HB₁, HB₂, HB₃, HB₄, HB₅, . . . HBₙ} depend on the intermediate outcomes in Hidden Layer A and the weights between them that arise from the training of the ANN. Finally, the Outputs {Out₁, Out₂, Out₃, . . . Outₙ} depend on the intermediate outcomes in Layer B and the weights that arise from the training of the ANN.

In one embodiment, the ANN uses uplift modelling, a technique to model the change in probability of an outcome resulting from an action. In an embodiment for TCM, the initial probability of hospital readmission is calculated based on the patient characteristics. Then one or more interventions (services) are applied, and the readmission probability is recalculated. In an iterative process, different interventions are added (or subtracted), and the ANN settles on the interventions which minimizes the probability of readmission.

In this embodiment, the inputs {In₁, In₂, In₃, In₄, . . . Inₙ} include patient characteristics patient outcomes, available services, cost of services, and location information. The outputs {Out₁, Out₂, Out₃, . . . Outₙ} are the priorities of intervention—i.e.: weights to the service selection module with the service that has the highest impact on reducing risk of readmission having the highest weight.

The outputs can also include the relative timing of intervention—that is, straightforward weighting does not account for the sequence of interventions. In a simple example, ordering transportation cannot be done until a follow up visit is scheduled and confirmed.

The outputs can also include an estimate of the cost effectiveness of intervention which can be used as another input to prioritize services. For example, certain drugs to treat diabetes are relatively new and expensive, whereas there may be inexpensive generic drugs with similar clinical performance.

In one embodiment, the ANN is initially trained using medical records e.g.: obtained from an EMR. The records may be curated e.g.: to ensure complete data without obvious errors. For instance, the ANN may be initially trained on previous instances of input data (e.g., a data set associated with an individual patient) and may be adapted to learn when data has been corrected (which may require further reference to a curated input data set). As one example, an input data set might include the following inputs, which would be indicative of obvious errors that might have been corrected later: Sex=Male, Date of Last Menstruation=30 Days Prior, Age=9, Number of Children=2. Alternatively, or in addition, the input data set might include more subtle errors such as inconsistent biomarker data, which the ANN might learn to recognize. Accordingly, the AI system may be adapted to provide ongoing and substantially real-time error-checking of input data on a large scale in a manner that could not be manually performed by a human due to the enormous amount of data involved in executing the disclosed systems and methods at scale with respect to ECM, TCM, or other care management environments. Training methods for the ANN can include supervised learning in which the curated data from the EMR is used. The ANN can be trained using back propagation, in which the input data are derived from the EMR and other databases, the known interventions which produce known results are compared to the ANN's output, and the weightings for intermediate nodes are adjusted to minimize error between the observed results and the ANN's results.

Like all AI systems, the reliability and usefulness of the outputs depends on the training dataset. In this invention, the training dataset can be obtained from multiple examples of patient data in the EMR from one or more HCS. A curated training dataset will use patient characteristics (as described above), interventions, and patient outcomes (among others) to train the AI system. In a preferred embodiment, the AI system will be continuously self-improving using data derived from new patients. U.S. Pat. No. 11,100,373 describes one example of an Autonomous and Continuously Self-Improving learning system.

In a preferred embodiment, the Prioritization module 203 can operate with sparse data (i.e.: less than complete data in the Input Layer 501) such that the services selected are the “best estimate” based on the data available.

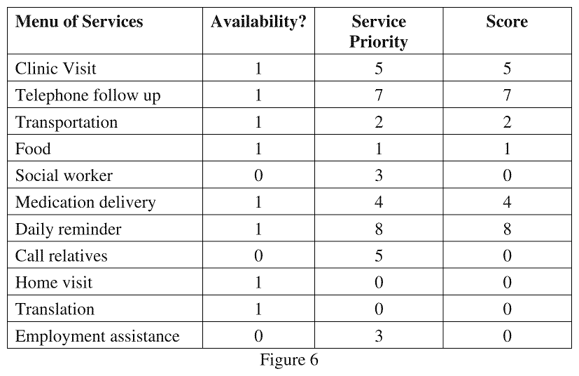

FIG. 6 shows an example of the operation of the Service Selection Module. The Menu of Services provided to the Service Selection Module is tagged with a modifier that reflects the availability of the service based, for example, on location or time (e.g.: a social worker may be available only on some days, whereas a telephone follow up is available 24/7). In this example, the modifier is one if the service is available, and zero if it is not. The Service Priorities 204 from the Prioritization Module 203 are modified by the availability of Services to generate a score of Recommended Services 206 to the Sequencing Module 207. In this example, the Recommended and Sequenced Services (ranked high to low) are Daily Reminder, Telephone follow up, clinic visit, medication delivery, transportation and food.

As shown in FIG. 2, this list is provided to the Central Service Provider 210 which coordinates the provision of services. For example, the CSP care coordinator may call the clinic to schedule a visit, arrange transportation for the client, initiate a daily reminder (e.g.: via mobile phone), call the pharmacy to order medication delivery, and then call the client to advise what action has been taken, notify of the date of the clinic visit and that transportation has been arranged and ask if food delivery is needed.

In one example, the patient suffers from advanced heart failure (HF). Following hospital discharge, the Service Selection Module recommends daily telephone follow-up by a medical assistant for the first week, followed by weekly follow-up thereafter. In addition, an automatic message is delivered to the patient's phone to remind the patient to take the prescribed medications at the same time every day. The message could be delivered via text message (e.g.: SMS, WhatsApp), by automated voice message, or personal contact with the Care Coordinator. In addition, based on the patient's social situation, in-home food delivery is scheduled automatically for every evening. In addition, the patient is provided with a “smart” bathroom scale that communicates with the Patient Outcomes module 213, and the patient is reminded to weigh himself at the same time every day e.g.: upon waking in the morning. In addition, the patient has a questionnaire pushed to his mobile phone to ask questions about his symptoms. After one week, the system records that the patient's weight is increasing, and he reports shortness of breath. Based on these new data points, the Prioritization module adjusts the services recommended and for example may recommend an in-home visit by a healthcare professional, medication adjustments, clinic visit, or alteration of exercise regime.

FIG. 3 is a representation of one embodiment of the organization of the CSP 300.

The CSP 300 coordinates the provision of services 308 to the patient 307 from the CSP. In some cases, the CSP may not have the resources to provide certain services, and they may be provided by an External Network of Services 309 to the patient 310. For example, if one of the prioritized services is telephone counseling in the patient's first language, then the CSP might employ telephone counselors fluent in that language from the External Network of Services 309. If one of the required services is transportation for clinical follow-up visits, then the CSP might arrange transportation services as needed.

In a preferred embodiment, the CSP 300 establishes contracts with the external network of services to predetermine availability, pricing, and quality.

In a preferred embodiment, the system is implemented on a cloud-based computing and data storage module shown as 301. Cloud-based services allow access to the system from virtually anywhere, at any time, e.g.: from a desktop computer or a smartphone app, offering flexibility for healthcare providers to monitor the progress of any patient regardless of the patient's location. The cloud database is updated in real time as new data become available. Of course, local computing and storage are also used as necessary for example personal computers, tablets, and smart phones.

In an alternative embodiment, the computing and storage module could be proprietary and local to the CSP, with remote access, or some combination of local and cloud based.

At the heart of the CSP are the Care Coordinators 302. The Care Coordinators are like the “traffic cops” of the system and are used to coordinate internal and outsourced services and provide certain services themselves. The Care Coordinators coordinate care through a network of care providers 303. The care coordinators may be co-located in a central office or may work remotely.

The care providers 303 can be full-time or part-time, and made up of a combination of at least one of the group of direct employees 304 (so-called “W2 employees” because they are on payroll), contract employees 305 (so-called “1099 employees” because they are treated as independent contractors), and occasional contractors 306 who might bid on jobs from time to time (the so-called “gig economy”). Care Coordinators may be trained as Medical Assistants, Nurse, or similar allied health professionals.

All operations of the CSP are underpinned by a Quality Management System (QMS) 312. The QMS specifies procedures, practices, and training (among other things) to ensure the quality of delivered services. For example, each of the W2 Employees 304, I099 Employees 305 and Occasional Contractors 306 are required to undergo rigorous training and assessment by the CSP before being allowed to provide services (a process commonly referred to as “onboarding”). Refresher training and assessment is provided from time to time.

The QMS will also impose certain quality standards on the external Network of Services. For example, the contract between the CSP and an external transportation company may require that the transportation must be available within 30 minutes of ordering, or the provider will risk a financial penalty.

When the CSP 300 (or 210 in FIG. 2) is presented with the Recommended and Sequenced Services 208 from the Service Selection Module 205 via the Sequencing Module 207, the Care Coordinator 302 assigned to that patient orders those services to be provided by at least one of the employee care providers 304 or 305.

If the Care Coordinator 302 determines that the required services are not within the range of expertise and available personnel, then the CSP will communicate a Service Request 311 to its network of occasional contractors 306 who are invited to bid to provide the requested services. One or more of the occasional contractors may offer to provide the requested services, and the Care Coordinator accepts at least one of the offers. Once the Care Coordinator knows the requested services can be provided, that availability is then communicated to the scheduling and follow-up module 213. In a preferred embodiment, the process of invitation to bid and acceptance of a bid is automated via the cloud computing platform, which unburdens the Care Coordinators and frees them up to provide more services.

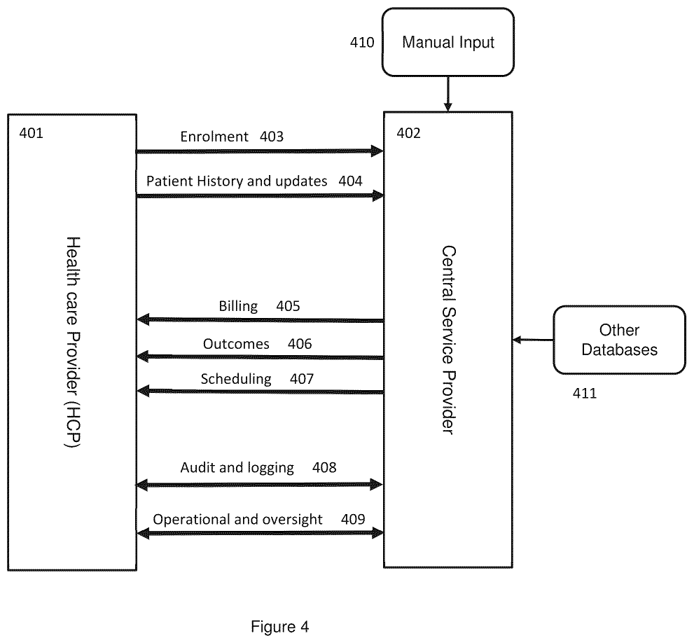

FIG. 4 is a representation in one embodiment of the data flows between the Health Care Provider (HCP) 401 and the CSP 402. The HCP could typically be a hospital (or network of hospitals), a clinic, a health care system and payer, a network of physicians, a community-based health care services organization, or a company that provides its own health care services. The CSP is the organization that provides and manages the system of the present invention.

In one embodiment, the HCP 401 initiates the communication by providing patient enrolment data 403 to the CSP 402. In other embodiments, the CSP may engage with multiple HCPs to sell its services.

Once the enrolment is accepted by both parties and the patient, the HCP 401 sends patient history data 404 to the CSP 402. As services are provided and the patient re-engages with the HCP, updates to the patient history are communicated to the CSP.

Once the CSP engages with the patient and initiates ECM services, the CSP communicates billing information 405 to the HCP (to get paid for its provision of services), outcomes data 406, and scheduling information 407 e.g.: for follow-up clinic visits.

Each of the HCP and the CSP will have an underlying Quality Management System (QMS) which requires the exchange of audit and logging information 408. The HCP is responsible for the patient's healthcare (the CSP provides services but does not take clinical responsibility), and thus there is an exchange of operational and oversight data 409 between the parties.

Once patient history and other information is communicated from the HCP to the CSP, the CSP may flag missing data. In that case, Manual Input 410 may be needed or helpful to augment the data obtained from the HCP.

The system may communicate information from Other Databases 411, for example from a social services provider.

The system will consolidate all the information available into Patient Characteristics 202 as input to the Prioritization Module 203.

As will be apparent to those skilled in the art, there will be multiple layers of data communication between the parties. The figures provided are a representation of one embodiment, and other embodiments are possible with additional or alternative data communication.

The description herein describes an ECM and TCM system, with one of the purposes to reduce hospital readmission. However, the system is not limited to dealing with patients after discharge from hospital. The system can be used for “hospital at home” or long-term enhanced care of chronic patients. In one embodiment, the system is used to provide enhanced care management for indigent people who might normally be eligible for a state's Medicaid services. Such patients are notoriously difficult to manage, for example the patient may be homeless or itinerant. The system may be used to improve the long-term health of this type of patient, thereby reducing the burden on a state's health care system. The system may also be used to support hospitals in the care of patients with “hospital at home” care delivery. Furthermore, the system can provide information that enables the state to better deliver health care services to its population at optimal cost.

In an alternative embodiment, the output of the Service Selection Module is reviewed by one or more persons (e.g.: physicians or medical assistants) who may override or re-order the selected services for the patient. For example, a physician familiar with the patient may be aware that the patient has an adversarial relationship with nearby family members and may therefore de-emphasize family education in the Selected Services. Any such reorder or override will be recorded and incorporated in the Prioritization module.

In a further embodiment, the system can determine the risk of hospital readmission prior to intervention, and then use the recommended services and historical knowledge to determine the risk (probability) of hospital readmission after provision of the services. For example, a Bayesian statistical approach can use a priori probability of readmission, modified by interventions to yield an updated probability of readmission.

The updated probability can be used in an iterative process to adjust the recommended services. In an example, the a priori probability of 30-day readmission is 30% for a patient, and with the suggested interventions it falls to 20%. The target is 10%, so in an iterative manner, the Prioritization module and service selection module adds services until the post-intervention probability of readmission reaches 10%. Similarly, the Prioritization module can automatically test if services make no difference to the updated probability of readmission, and not include those services in the recommended and sequenced services, thereby reducing useless time, cost, and effort.

This iterative process can be used in a business pricing model. For example, many patients for whom this system and method is suitable are those covered by a value-based reimbursement system in which the HCP is paid a fixed fee per enrolled patient per month (PEPM). Medicare Advantage and Medicaid are examples of a value-based reimbursement approach.

In a real-world situation, the CSP must also be financially viable. With a value-based reimbursement system and a fixed fee PEPM, the CSP 107 needs to be careful that the financial risk is not transferred to the CSP from the HCS 102. In one embodiment, prior to accepting enrolment of a patient, the CSP will evaluate the a priori risk (probability) of hospital readmission, the post-intervention risk (probability) of hospital readmission, and the anticipated cost of providing the services. If the cost exceeds the PEPM fee for that patient, the CSP 107 may decide to reject the enrolment of that patient.

The CSP has access to large amounts of data on each patient and the population of patients. Those data have intrinsic value and can be sold for example to government organizations responsible for making policy.

The CSP has access to data from each patient, some of it in real time or near real time. That data can be used to generate an alarm. For example, a diabetic patient may regularly travel between home and work and have a follow up telephone call every few days. On one occasion, the call is not answered. The Care Coordinator initiates a Database query and learns that the patient has not been to work for the last few days, and the patient's telephone has not moved for two days (according to the GPS tracking). The Care Coordinator immediately calls a neighbor (registered as one of the available Services) to knock on the patient's door. When the neighbor reports the knock is not answered, the Care Coordinator initiates a call to emergency services who arrive and find the patient immobile on his bed in hypoglycemic shock. Intravenous glucagon is administered, and the patient recovers without further incident. In this example, had the patient not been treated as quickly, a repeat hospitalization might have been needed.

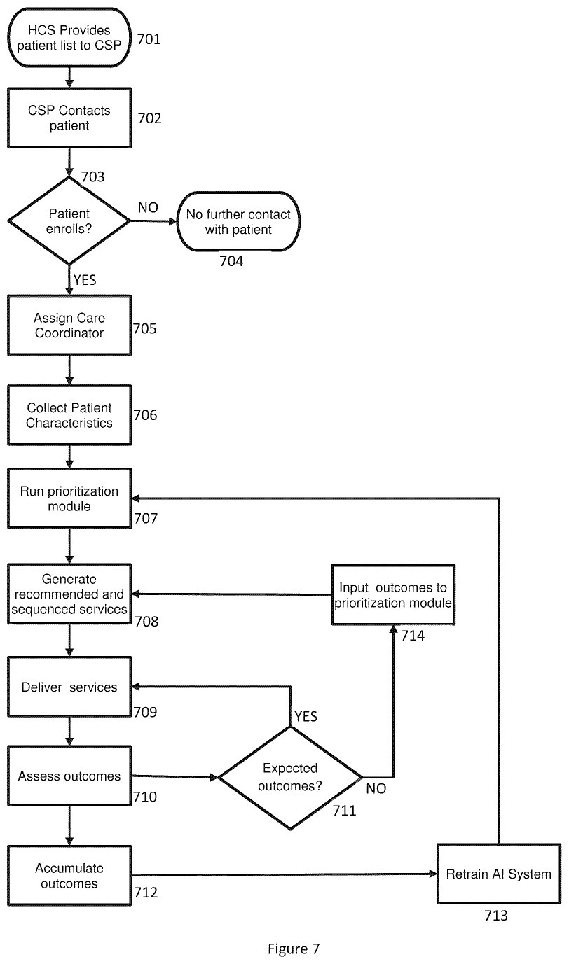

An example of the method with a typical patient is illustrated in FIG. 7.

Before the process starts, the HCS contracts with the CSP to set a price for the CSP to provide the services. This is usually based on a fixed fee Per Enrolled Person Per Month (“PEPM”). The contract is usually for a fixed period of time e.g.: annually. In another embodiment, the CSP may use risk-based pricing to the HCS and accept lower-risk patients at a lower fee, thus enabling all parties to optimize their financial position (profit).

The process begins with a HCS providing a list of potential patients to the CSP 701. The CSP then contacts 703 each potential patient to invite the patient to enroll in the service. Not all patients may choose to be enrolled, and the CSP may not wish to enroll some patients e.g.: those with so many health problems that the CSP may struggle to provide the resources needed. The CSP may use an external organization to help enroll patients. Patients who decline enrollment 704 will not be contacted again for the duration of the current contract.

Patients who decline enrollment who are still managed by the HCS may serve as a “control group” for comparing outcomes with and without the services ECM and TCM provided by the CSP. Patient swho decline enrollment may be eligible for enrolment in the next contract cycle, and may be contacted again by the CSP.

Once a patient is enrolled, the CSP assigns a care coordinator to that patient 705. The patient characteristics are collected 706 e.g. from the EMR and other sources. The patient characteristics are used as the input to the prioritization module 707 as described above and illustrated in FIG. 2.

From that, the prioritization module generates the recommended and sequenced services 708 which are then delivered 709, as organized by the Care Coordinator.

Outcomes are assessed 710 from time to time e.g.: on a regular follow up schedule, in conjunction with a patient contact (e.g.: clinic visit) or triggered by an unscheduled event e.g.: hospital admission. Those outcomes are assessed 711. If the outcomes are as expected e.g.: weight or blood pressure reported as normal during a follow-up visit, then the services continue to be delivered. If the outcomes are not as expected (e.g.: weight gain, high blood pressure) then those unexpected outcomes are fed back 714 to the prioritization module which may then generate different recommended and sequenced services 708.

Over time, outcomes are accumulated 712 from many patients. Those accumulated outcomes can be used to retrain the AI system 713 which improves the prioritization module.

As will be apparent to a person skilled in the art, the embodiments described herein are illustrative but not limiting of the principles of the present invention. The present invention is not limited to the examples provided, embodiments described, and/or the description, but rather only by the scope of the allowed claims.

1. A computerized data management and processing system configured to interact with a health care system (HCS) and a central service provider (CSP) to provide data pertaining to transition care management (TCM) or extended care management (ECM) of a patient, the system comprising:
at least one computing device in communication with the HCS and the CSP, the at least one computing device comprising:
a prioritization module,
memory configured to store computer-executable instructions; and
at least one processor configured to access the memory and execute the computer-executable instructions to:
receive service data comprising a menu of services indicative of one or more services associated with the HCS, the one or more services comprising service location and service time data;
receive, from the HCS, patient data comprising one or more patient characteristics associated with the patient and historical data associated with a patient population comprising at least the patient, the one or more patient characteristics comprising location and time data;
cause the prioritization module to generate, based on the service data and the patient data, service priority data comprising one or more service priorities corresponding to the one or more services and associated with the patient;
determine, based on the service data, the patient data, and the service priority data, at least one recommended service of the one or more services to provide to the patient, the at least one recommended service associated with the TCM or the ECM of the patient;
generate, based on the service data, the patient data, the service priority data, and the determination of the at least one recommended service, recommended and sequenced service data comprising a recommended service sequence for providing the at least one recommended service to the patient;
transmit the recommended and sequenced service data to the CSP;
receive patient outcome data indicative of one or more patient outcomes associated with provision or attempted provision of the at least one recommended service to the patient;
cause the prioritization module to modify, based on the recommended and sequenced service data and the patient outcome data, the patient data and the service priority data;
predict, based on the patient data, a change in risk of hospital admission after provision of the at least one recommended service to the patient; and
automatically modify, based on the prediction of the change in risk of hospital admission, the recommended and sequenced service;
wherein the prioritization module comprises an artificial neural network (ANN), the ANN comprising an input layer, a hidden layer, and an output layer, the ANN trained using curated medical records in order to provide real-time error checking of patient data.

2. The system of claim 1, wherein the location and time data is indicative of the permanent location or the current location of the patient during at least a first time.

3. The system of claim 2, wherein the at least one processor is further configured to access the memory and execute the computer-executable instructions to:
receive service availability data indicative of a geographical availability and a time availability of the one or more services;
determine, based on the location and time data and the service availability data, one or more available services of the one or more services to provide to the patient; and
modify, based on the determination of the one or more available services, the service data.

4. The system of claim 2, wherein the location and time data is indicative of the current location of the patient at the first time, the current location transmitted in substantially real time by a mobile device associated with the patient.

5. The system of claim 1, wherein the CSP is associated with a care coordinator, the at least one processor further configured to access the memory and execute the computer-executable instructions to:
receive, from the care coordinator, override or re-order instructions associated with the one or more service priorities; and
modify, based on the override or re-order instructions, the service priority data.

6. (canceled)

7. The system of claim 1, wherein the one or more patient characteristics comprise at least one clinical characteristic and at least one social characteristic of the patient, and each of the one or more services is categorized as a clinical service or a social service in the menu of services.

8. The system of claim 1, wherein at least one of the one or more services comprises an automated communication between the CSP and the patient.

9. The system of claim 8, wherein the automated communication is automatically transmitted by a smart device in communication with the at least one computing device and configured to provide a clinical service or a social service to the patient, the clinical service or the social service comprising transmission of the automated communication, the at least one processor further configured to access the memory and execute the computer-executable instructions to:
modify, based on the provision of the clinical service or the social service by the smart device, the patient outcome data.

10. The system of claim 1, wherein the at least one computing device is further in communication with one or more external care or service providers, the at least one processor further configured to access the memory and execute the computer-executable instructions to:
receive external provider data from the one or more external care or service providers; and
modify, based on the external provider data, the service priority data.

11. The system of claim 10, wherein the at least one processor is further configured to access the memory and execute the computer-executable instructions to:
automatically invite at least a portion of the one or more external care or service providers to bid to provide at least one of the one or more services;
receive one or more bids to provide at least one of the one or more services from at least the portion of the one or more external care or service providers; and
automatically accept or reject the one or more bids.

12. The system of claim 1, wherein the at least one computing device is in communication with at least one of the HCP or the CSP via a cloud platform, and at least one of generating the service priority data, generating the recommended and sequenced service data, or modifying the patient data and the service priority data is automatically performed in substantially real time via the cloud platform.

13. The system of claim 12, wherein the ANN further comprises a second hidden layer.

14. The system of claim 1, wherein the ANN uses an uplift modeling technique.

15. A method of computerized data management and processing of data associated with a health care system (HCS) and a central service provider (CSP) to provide data pertaining to transition care management (TCM) or extended care management (ECM) of a patient, the method comprising:
receiving, by at least one computing device in communication with the HCS and the CSP, service data comprising a menu of services indicative of one or more services associated with the HCS, the one or more services comprising service location and service time data, and the at least one computing device comprising a prioritization module;
receiving, by the at least one computing device, from the HCS, patient data comprising one or more patient characteristics associated with the patient and historical data associated with a patient population comprising at least the patient, the one or more patient characteristics comprising location and time data;
generating, by the prioritization module, based on the service data and the patient data, service priority data comprising one or more service priorities corresponding to the one or more services and associated with the patient;
determining, by the at least one computing device, based on the service data, the patient data, and the service priority data, at least one recommended service of the one or more services to provide to the patient, the at least one recommended service associated with the TCM or the ECM of the patient;
generating, by the at least one computing device, based on the service data, the patient data, the service priority data, and the determination of the at least one recommended service, recommended and sequenced service data comprising a recommended service sequence for providing the at least one recommended service to the patient;
transmitting, by the at least one computing device, the recommended and sequenced service data to the CSP;
receiving, by the at least one computing device, patient outcome data indicative of one or more patient outcomes associated with provision or attempted provision of the at least one recommended service to the patient;
modifying, by the prioritization module, based on the recommended and sequenced service data and the patient outcome data, the patient data and the service priority data;
predicting, by the at least one computing device, based on the patient data, a change in risk of hospital admission after provision of the at least one recommended service to the patient; and
automatically modifying, by the at least one computing device, based on the prediction of the change in risk of hospital admission, the recommended and sequenced service data,
wherein the prioritization module comprises an artificial neural network (ANN), the ANN comprising an input layer, a hidden layer, and an output layer, the ANN trained using curated medical records in order to provide real-time error checking of patient data, and
wherein the at least one computing device is in communication with at least one of the HCP or the CSP via a cloud platform, and at least one of generating the service priority data, generating the recommended and sequenced service data, or modifying the patient data and the service priority data is automatically performed in substantially real time by the at least one computing device via the cloud platform.

16. The method of claim 15, wherein the location and time data is indicative of the current location of the patient, the method further comprising:
transmitting, by a mobile device associated with the patient, in substantially real time, the current location of the patient;
receiving, by the at least one computing device, service availability data indicative of a geographical availability and a time availability of the one or more services;
determining, by the at least one computing device, based on the location and time data and the service availability data, one or more available services of the one or more services to provide to the patient; and
modifying, by the at least one computing device, based on the determination of the one or more available services, the service data.

17. The method of claim 15, wherein the one or more patient characteristics comprise at least one clinical characteristic and at least one social characteristic of the patient, and each of the one or more services is categorized as a clinical service or a social service in the menu of services.

18. The method of claim 15, wherein at least one of the one or more services comprises an automated communication between the CSP and the patient, the method further comprising:
automatically transmitting, by a smart device in communication with the at least one computing device and configured to provide a clinical service or a social service to the patient, the automated communication;
providing, by the smart device, the clinical service or the social service to the patient, wherein the clinical service or the social service comprises transmission of the automated communication by the smart device; and
modifying, by the at least one computing device, based on the provision of the clinical service or the social service by the smart device, the patient outcome data.

19. The method of claim 15, wherein the CSP is associated with a care coordinator and the at least one computing device is further in communication with one or more external care or service providers, the method further comprising:
receiving, by the at least one computing device, from the one or more external care or service providers, external provider data;
modifying, by the at least one computing device, based on the external provider data, the service priority data;
automatically inviting, by the at least one computing device, at least a portion of the one or more external care or service providers to bid to provide at least one of the one or more services;
receiving, by the at least one computing device, from at least the portion of the one or more external care or service providers, one or more bids to provide at least one of the one or more services;
automatically accepting or rejecting, by the at least one computing device, the one or more bids;
receiving, by the at least one computing device, from the care coordinator, override or re-order instructions associated with the one or more service priorities; and
modifying, by the at least one computing device, based on the override or re-order instructions, the service priority data.

20. The method of claim 15, wherein the ANN further comprises a second hidden layer.

21. The system of claim 12, wherein at least one of receiving the service data, receiving the patient data, or receiving the patient outcome data is automatically performed in substantially real time via the cloud platform.
